(19) United States
(12) Patent Application Publication
(10) Pub. No.: US 2021/0348154 A1
(43) Pub. Date: Nov. 11, 2021

(54) HETERO-FUNCTIONAL COATING FOR CONJUGATING BIOMOLECULES ON A SOLID SUPPORT AND USE THEREOF FOR BIO ANALYSIS

(71) Applicant: Waters Technologies Corporation, Milford, MA (US)

(72) Inventors: Beatrice Muriithi, Attleboro, MA (US); Wenjing Li, Shrewsbury, MA (US); Oksana D. Tchoul, Winchester, MA (US); Matthew A. Lauber, North Smithfield, RI (US); Darryl W. Brousmiche, Grafton, MA (US)

(73) Assignee: Waters Technologies Corporation, Milford, MA (US)

(21) Appl. No.: 17/314,853

(22) Filed: May 7, 2021

Related U.S. Application Data
(60) Provisional application No. 63/022,051, filed on May 8, 2020.
(60) Provisional application No. 63/022,047, filed on May 8, 2020.

(51) Int. Cl. C12N 11/06; G01N 33/543

(52) U.S. Cl. CPC C12N 11/06; C12Y 304/00; C12Y 302/01; G01N 33/54306

(57) ABSTRACT

The present disclosure relates to a hetero-functional coating applied on a solid support. The coating includes a first functionality for conjugating biomolecules for the analysis of a protein or nucleic acid, and a second functionality for preventing undesired interactions between analytes of interest and the surface of solid support.

### CROSS-REFERENCE TO RELATED APPLICATIONS

This application claims priority and benefit to U.S. Provisional Patent Application No. 63/022,047 filed on May 8, 2020, entitled “Hetero-Functional Coating for Conjugating Biomolecules on a Solid Support and Use Thereof for Bioanalysis.” This application claims priority and benefit to U.S. Provisional Patent Application No. 63/022,051 filed on May 8, 2020, entitled “Hetero-Functional Coating for Conjugating Biomolecules on a Solid Support and Use Thereof for Bioanalysis.” The contents of each are incorporated herein by reference in their entirety.

### FIELD OF THE TECHNOLOGY

The present disclosure relates to a material for use in conjugating biomolecules. More specifically, the present disclosure relates to a coating, such as a hetero-functional coating, for conjugating biomolecules on a solid support surface and use thereof for bioanalysis.

### BACKGROUND

Biomolecules are complex molecules, which require complex workflows for analysis. These complex workflows involve numerous steps including various sample preparation steps, such as, sample clean up and protein digestion. Obtaining substantially complete digestion while not effecting sample quality (e.g., not introducing byproducts which require additional cleanup) is challenging. For example, some enzymes used in digestion when used in solution can become unstable, leading to byproducts. Immobilization of these enzyme helps to stabilize the enzymes. However, the conventional techniques used to immobilize enzymes leads to secondary interactions of the analytes of interest with the surface of the support, impacting digestion efficiency and sample recovery.

### SUMMARY

In general, the technology of the present disclosure is directed to a material for use in the analysis of biomolecules. More particularly, the technology is directed to the use of a hetero-functional coating that can be applied to a substrate, e.g., a solid support for conjugating biomolecules. The hetero-functional coating provides numerous advantages over conventional techniques used in processing biomolecules. The technology is directed to coatings used in the bioanalysis of complex samples and adapted to provide two or more functionalities (e.g., hetero-functional coating) to an underlying solid support. These functionalities are designed to provide improved efficiency to the bioanalysis workflow and improved recovery of the analytes. In particular, the hetero-functional coating provides the functionality of biomolecule conjugation while at the same time providing a functionality that eliminates or reduces secondary interactions during sample processing (e.g., digestion, affinity cleanup). In addition, the materials of the present technology are advantageous, as they are typically vapor or liquid deposited, to uniformly (i.e., no gaps or holes) coat the underlying solid support.

In one aspect, the present disclosure is directed to a hetero-functional coating applied on a solid support (e.g., a particle, membranes, a planar substrate, a portion of a vial, well plate, pipette tip, etc). The coating imparts a first functionality and a second functionality to the surface of the solid support. The first functionality is for conjugating biomolecules for the analysis of a protein or nucleic acid. The second functionality is for preventing undesired interactions between analytes of interest and the surface of the solid support. For example, the second functionality can be to impart hydrophilic properties to the surface of the support. The hetero-functional coating is able to uniformly coat the underlying solid support and provides a total surface coverage of at least 5 μmoles/m² or greater (e.g., 7 μmoles/m², 9 μmoles/m², 15 μmoles/m²). As this is a hetero-functional coating, there are at least two functionalities imparted to the surface of the underlying support. In some embodiments, the ratio of the second functionality to the first functionality is at least 15% of the total surface coverage (e.g., 18%, 20%, 30%, 45%, etc.).

In another aspect, the present disclosure is directed to using the hetero-functional coating applied to a solid support for the analysis of a biomolecule. In particular, the hetero-functional coating applied to one or more of the disclosed solid supports can be used in sample processing of a complex biological sample. For example, the hetero-functional coating applied to the solid support can be used with immobilized affinity ligands for an affinity processing step or with immobilized enzymes for a digestion or an enzyme catalyzed reaction step.

In another aspect, the present disclosure is directed to a method of coating a solid support. The method includes activating the solid support surface, depositing the coating on the solid support surface to form a surface coverage greater than 5 μmoles/m²; and processing the coating to form two portions: a first coating portion with functionality for bioconjugation and a second coating portion with a functionality to reduce undesired interactions. In general, the coating is deposited and then processed to provide the surface of the solid support with the two or more functionalities.

In another aspect, the present technology relates to a hetero-functional coating on a solid support surface (e.g., a particle, membrane, a microchip, a planar substrate, a glass slide, a PCR tube, a pipette tip, a multi-well plate, etc.). To create the hetero-functional coating (i.e., to form the two or more functionalities) the following steps are performed. First, the solid support surface is activated. Next, a coating including epoxide groups is prepared. The coating is deposited on the solid support surface to form surface coverage greater than 5 μmoles/m²; the epoxide groups are hydrolyzed or their rings are opened into diols to form a hydrophilic coating (e.g., a first functionality); and then the diols are oxidized into aldehyde groups (e.g., to provide the second functionality) in a controlled manner to form the hetero-functional coating. In some embodiment at least 15% (e.g., 15%, 17%, 20%, 25%) of the diol groups have been oxidized into the aldehyde groups.

The present technology includes other hetero-functional coatings made by other processes but still useful in the analysis of biomolecules. In another aspect, the present technology relates to a hetero-functional coating on a solid support surface. To create the hetero-functional coating (i.e., to form the two or more functionalities) the following steps are performed. First, the solid support surface is activated. Next a coating including epoxide groups is prepared. The coating is deposited on the solid support surface to form surface coverage greater than 5 μmoles/m²; the epoxide groups are hydrolyzed or their rings are opened into diols to form a hydrophilic coating on the solid support surface, wherein a percent diol on the solid support surface is greater than 25% (e.g., 30%, 35%, 40%, etc.). The process to create the hetero-functional coating can further include a step of activating the coating with one or more linker chemistries that carry functionality for conjugating biomolecules. This activation step can be a single step or a multi-step process using a plurality of hetero-functional molecules and homo-functional molecules reacted with the residue epoxide.

The present technology also encompasses other hetero-functional coatings and processes to create them. In one aspect, the technology relates to a hetero-functional coating on a solid support surface. To create the hetero-functional coating, the following steps are performed. First, the solid support surface is activated. Next a coating including diol groups is prepared. The coating is deposited on the solid support surface to form surface coverage greater than 5 μmoles/m²; the coating is activated with one or more linker chemistries by preparing the linker chemistries in a multi-step process using a plurality of hetero- and homo-functional molecules reacted to the aldehyde groups. In some embodiments the plurality of hetero- and homo-functional molecules are chosen or selected from any of the following: alkyldiamine, alkyl dihydrazide, diazides, bifunctional PEG diamines, bifunctional PEG hydrazide, multi arm PEG azides, multi arms PEG hydrazides, acrylate PEG amine, biotin PEG amine, thiol PEG amine, or derivatives thereof.

The present technology is also directed to apparatus that include the hetero-functional coating on a solid support. In some embodiments, a coated solid-support substrate is positioned within the interior of a vessel which is designed to receive the sample for processing. In other embodiments, the hetero-functional coating is deposited on a sample receiving portion of the processing apparatus.

In general, the materials and methods provided in accordance with the present technology provide numerous advantages. For example, the materials provide a stable platform for the processing of a biomolecule, while at the same time preventing undesired secondary interactions between the analytes and the solid support. As a result, increases in efficiency (e.g., elimination of further clean up or processing steps, increase in digestions by reducing the % missed cleavage) as well as recovery (e.g., elimination of secondary interactions) can be achieved. In addition to increases in efficiency and recovery, other advantages are possible such as increase in thermal and chemical stability allowing the bioconjugate biomolecule to be used in different conditions. For example, the materials and methods of the present technology can be tailored to accommodate various forms and apparatus. In particular, the coating can be applied and tailored to provide an optimal result. That is, the coating can uniformly coat any underlying substrate including various apparatus used for processing a sample. In addition, the ratio of the second functionality to the first functional can be tuned or adapted to a particular biomolecule processing step (e.g., digestion of a particular protein).

### BRIEF DESCRIPTION OF THE DRAWINGS

The technology will be more fully understood from the following detailed description taken in conjunction with the accompanying drawings, in which:

FIG. 1 is a flow chart illustrating a method, in accordance with the present disclosure.

FIG. 2A displays an example diol bonding process.

FIG. 2B displays an example coating on a particle or surface.

FIG. 2C displays an example coating on a particle or surface.

FIG. 3 displays an example device with a hetero-functional coating to conjugate biomolecules.

FIG. 4A is a NIST mAB peptide map for an in-solution trypsin digestion example.

FIG. 4B is a NIST mAB peptide map for a SMART Digest™ example.

FIG. 4C is a NIST mAB peptide map for a prototype, in accordance with the present disclosure.

FIG. 5 is a bar graph displaying the sequence coverage in accordance with the present disclosure.

FIG. 6 is a bar graph displaying the percentage missed cleavage in accordance with the present disclosure.

FIG. 7 is a flow chart illustrating the peptide mapping workflow.

FIG. 8 is a flowchart of an example of affinity current workflow.

FIG. 9 is a flowchart of an example of trypsin digestion current workflow.

FIG. 10 is an infographic of challenges with immobilized affinity ligands and enzymes.

FIG. 11 is a graph of thermograms including a comparison between the present disclosure and SMART Digest™.

FIG. 12 is a graph of a peptide recovery including a comparison between the present disclosure and SMART Digest™.

FIG. 13 is a graph of released trypsin including a comparison between the present disclosure and SMART Digest™.

### DETAILED DESCRIPTION

Protein-based therapeutics have become an important class of medicines for incurable diseases. Similar to small molecule drugs, protein-based drugs must be extensively characterized. The critical quality attributes of protein-based drugs must be fully determined to ensure safety and efficacy. Compared to small molecule drugs, protein-based drugs are large and complex. In some examples, complex workflows are required to analyze these complex molecules. Thus, the protein-based drugs can be difficult to characterize and analyze.

Advancements in mass spectrometry have made liquid chromatography/mass spectrometry methods (LC/MS) an important technology to characterize and analyze protein-based drugs. LC/MS characterization of biomolecules, such as proteins, requires several steps, including sample cleanup with affinity devices followed by digestion to reduce biomolecules to subunits that can easily be separated and analyzed. Consequently, sample prep (requiring clean-up and digestion steps) can be a significant part of workflows for analysis of biomolecule samples.

Current sample preparation workflow for protein-based drugs is time consuming, which sometimes results in sample loss, inconsistent results, and irreproducibility. For example, sample cleanup requires efficient affinity devices with lower secondary interactions, higher specificity, and higher capture efficiency. On the other hand, faster, less sample loss and minimal post transformation modification as well as complete digestion are preferred for the digestion step.

Some example solutions to these challenges include using affinity devices with immobilized ligands for sample clean-up and digestion with solution enzymes. Solution enzymes such as trypsin can be used for digestion of biotherapeutics. However, workflow based on solution trypsin can be time consuming and sometimes requiring sample cleanup, which may result in sample losses. In addition, solution enzyme workflows may be restricted to offline and cannot be easily transformed to online working. Solution enzymes such as trypsin have thermal and chemical limitations. Immobilized enzymes offer several advantages over solution enzymes. But immobilized enzymes full potential is yet to be realized.

For example, some solutions include affinity ligands immobilized on agarose or polymer beads, which can have mechanical stability limitations. Solution enzyme digestion protocols are commonly used for digestion of biotherapeutics for analysis with LC/MS. Immobilized enzymes offer several advantages over solution enzymes such as increased temperature and chemical stability. However, use of these products is limited due to sample loss and incomplete digestion as a result of secondary interactions.

In one example, the current status workflow includes: protein clean-up, denature, reduction, and alkylation; digestion; quenching peptide clean-up; and LC/MS.

SMART Digest™ (available from Thermo Fisher Scientific, Waltham, Mass.) is a product based on trypsin immobilized on a polymer particle. These particles have pressure limitations. In addition, the process of immobilization can only be used on polymer particles, not on silica or inorganic surfaces or particles or device surfaces. In addition, SMART Digest™ suffers secondary interactions that results in higher missed cleavages, inconsistent results, and lower sample recovery.

Immobilization of enzymes and affinity ligands on solid support or surfaces with functionalities described in the present disclosure will result in an increase in their applicability, by increasing stability and eliminating secondary interaction with the analytes.

For digestion of protein samples, the present disclosure solves the problem of secondary interactions between hydrophobic peptides generated after digestion and the solid support surface of immobilized enzymes. When comparing the present disclosure to solution enzymes (more specifically trypsin), solution enzymes suffer from autolysis, thermal instability, and chemical instability. Immobilization of enzymes helps to stabilize the enzymes against these conditions. However, most immobilized enzymes suffer secondary interactions which impacts the digestion efficiency reproducibility and sample recovery. The coating of the present disclosure reduces secondary interactions and the negative impacts of secondary interactions resulting in consistent high digestion efficiency.

Specifically, the present disclosure is a hetero-functional coating with two parts. One part of the coating has functionality to attach and conjugate a biomolecule (e.g., an enzyme or affinity ligand) and a second part of the coating has functionality to eliminate secondary interactions. The second part of the coating can include a hydrophilic group to eliminate the undesired secondary interactions between the solid support surface and the analytes of interest.

The coating of the present disclosure is deposited on a solid support and processed in a controlled manner to yield both functionalities. For affinity devices, the coating can be applied on different surfaces, such as particles, membranes, monoliths, a surface of a device, I microchip channel or any surface of any material type (of polymer or silica) and on different types of materials, such as polymers, silica, inorganic, or any combinations. Due to the reduced secondary interactions, recoveries of the analyte of interest, such as proteins, can be increased.

The coating of the present disclosure repels analytes of interest off the underlying solid surface during sample processing. Some of the coating properties include thicker hydrophilic coatings, functional groups on the surface, and coating versatility for different surfaces. The coating properties help reduce or eliminate secondary interactions, allowing enzyme and affinity ligands to perform at full potential. By operating at full potential, the data quality is improved, e.g., increased sequence coverage and decreased missed cleavages and increases sample recovery. In some examples, the coating is an epoxide-based coating. The coating can be a hetero-functional coating, e.g., an epoxide group and a diol group. The epoxide group can bioconjugate biomolecules and activate into different linker chemistry. The diol group can eliminate undesired interactions of the sample with the surface.

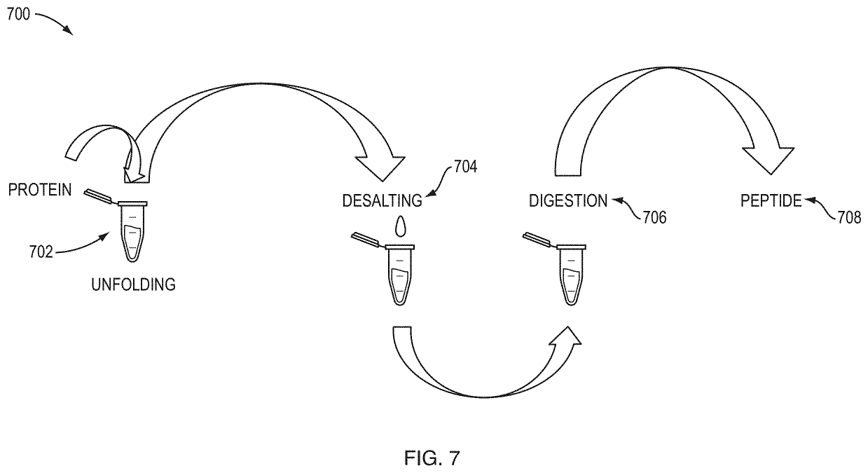

FIG. 7 is a flow chart illustrating the peptide mapping workflow 700. In some examples, peptide mapping workflow 700 includes four parts. A part one 702 includes a sample with an analyte of interest, such as a protein, is unfolded. A part two 704 includes desalting the sample to remove reagents used in the unfolding step, which includes the unfolded analyte of interest. A part three 706 includes digesting the analyte of interest of the sample with choice of enzyme. Here, the device used in digesting the analyte of interest includes a hetero-functional coating of the present disclosure. After the analyte of interest is digested, a part four 708 includes collecting the sample with digested analyte of interest for analysis with downstream processes.

In some examples, part one 702 and part two 704 can be dependent on the analyte of interest. For example, part one 702 and part two 704 can be considered pre-treatment steps and may not be required based on the analyte of interest, such as a protein.

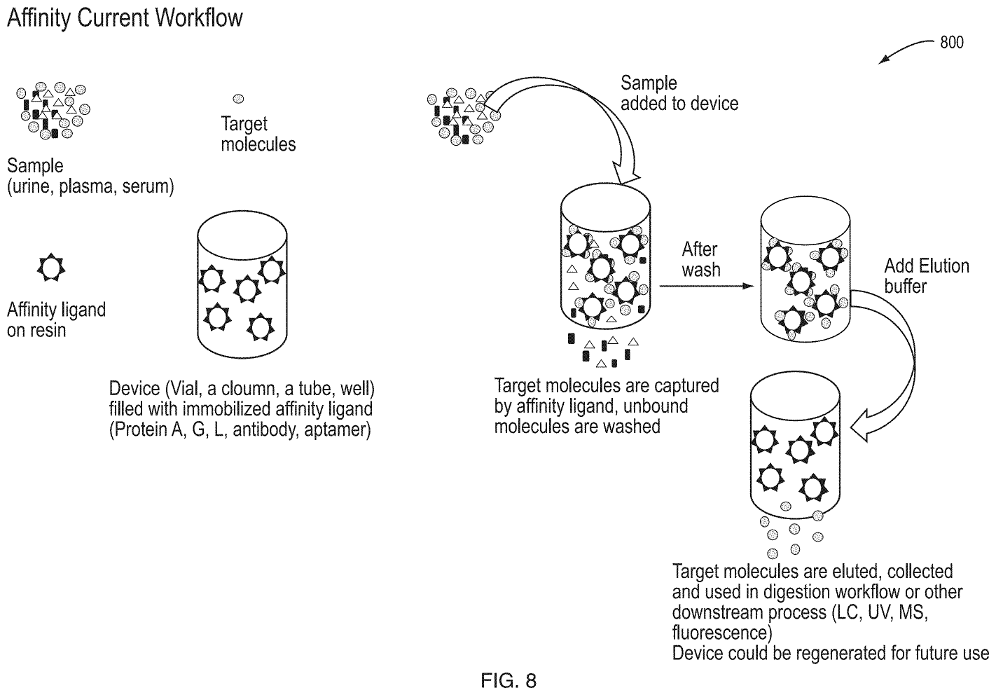

FIG. 8 is a flowchart of an example of affinity current workflow 800.

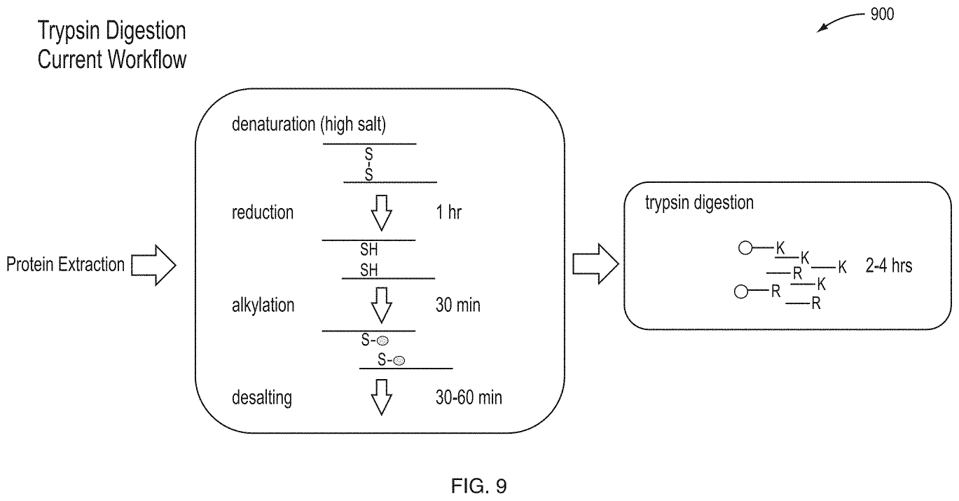

FIG. 9 is a flowchart of an example of trypsin digestion current workflow 900. The focus of the present technology is to improve the digestion step by eliminating undesired interactions.

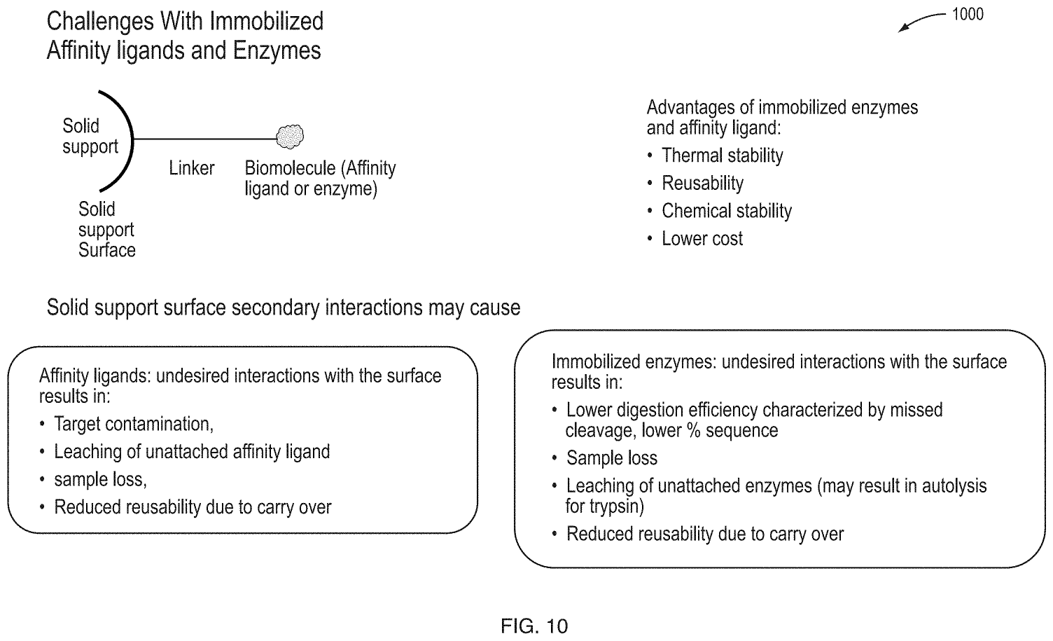

FIG. 10 is an infographic 1000 of challenges with immobilized affinity ligands and enzymes.

To address these challenges, the present technology provides a new material tailored to increase sample processing efficiency together with sample recovery. The material includes a hetero-functional coating applied to a solid support.

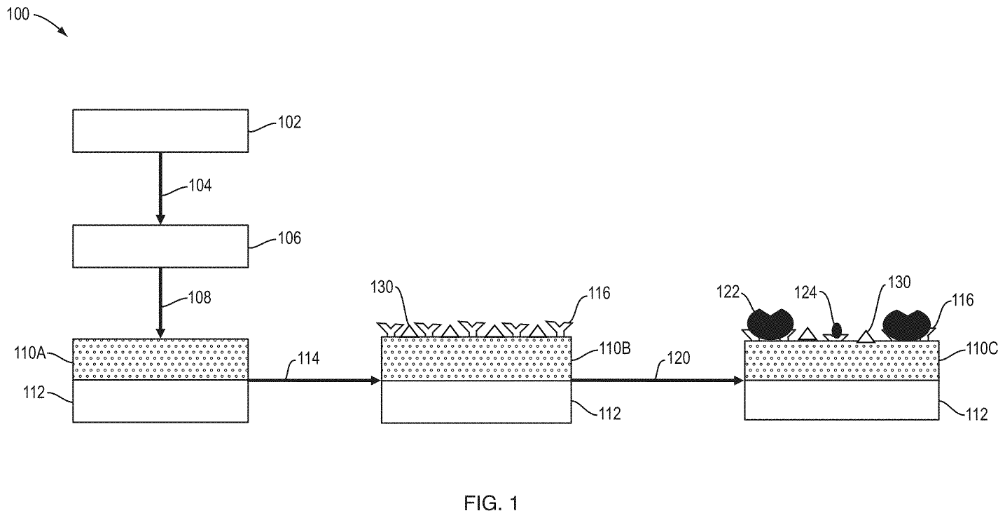

FIG. 1 discloses a system 100 with a solid surface 102, an activated surface 106, a coating 110A coating with hydrophilic surface groups 130, a support surface 112, a hydrophilic coating 110B including a functionality for bioconjugation 116 and hydrophilic surface groups 130, a hydrophilic coating 110C with hydrophilic surface groups 130 and functionality for bioconjugation 116 attached to a biomolecule 122 or a quencher (an end cap) 124. Coating 110A, hydrophilic coating 110B, hydrophilic coating 110C are collectively referred to as coating 110.

Coating 110 of system 100 can be prepared in a multistep process. A first step 104 activates solid surface 102 to be activated surface 106 to be able to receive a coating. Numerous methods can be used to activate solid surface 102 as long as the method prepares solid surface 102 to be receptive to coating. For examples, solid surface 102 can be activated by processes such as, and not limited to, solution etching, plasma, molecular vapor deposition of active groups, chemical vapor deposition of active groups, gas phase etching, polymerization, and surface bonding with active functionalities.

Solid surface 102 can be numerous different types of solid surfaces. For example, solid surface 102 can include the surface of particles, resins, monoliths, membrane, devices, or channels on a chip or a microchip. In some examples, the surface of the devices is an interior surface of a vial, tube, well, pipette, or glass slide. In some examples, the surface of the particles can be nonporous particles, superficially porous, or fully porous particles. In some examples, the particles are paramagnetic due to a paramagnetic core, paramagnetic shell or a mixture thereof.

Activated surface 106 forms support surface 112 when coating 110A is added. A second step 108 includes depositing coating 110A on support surface to form surface coverage of support surface. In some examples, depositing coating 110A on support surface 112 includes preparing coating 110A by pre-polymerizing an alkoxy silane containing an epoxide and reacting the pre-polymer with the solid support surface.

The alkyloxy silane can be an epoxide containing silane selected from 5.5 epoxyhexyltrifuntional silane, 3 glycidopropyltrifunctional silane or 2-(3,4-Epoxycyclohexyl) ethyltrifunctional silane. The alkoxy silane can also be an carboxy silane n-hydroxysuccinimide ester. Conjugating the biomolecules can include conjugating the biomolecules through the carboxyl n-hydroxysuccinimide ester via substitution reaction. The coating process of second step 108 can include using an organic monomer and polymerization techniques. The organic molecules can be chosen from a group, the group including glycidol, phenylglycidol and derivatives, trimethylpropanetriglycidyl ether, tris(4-hydroxyphenylmethani triglycidyl ether, PEG diglycidyl ether, ethylene glycol diglycidyl ether, glycidyl glycerol (triglycidyl) ether polyfunctional, trimethylolpropane triglycidyl ether, bisphenol A diglycidyl ether and derivatives. Using monomers and polymerization techniques includes covalently bonding coating 110A on support surface 112, such as a polymer surface. Coating 110A can be adsorbed on support surface 112.

A third step 114 activates coating 110A to have hydrophilic surface groups 130 as well as functionality for bioconjugation 116. In some examples, functionality for bioconjugation (first functionality) 116 is a first functionality for conjugating biomolecules for application in analysis of a biotherapeutic, and hydrophilic surface groups (second functionality) 130 is a second functionality for preventing undesired interactions between analytes of interest and the surface of solid support. In some examples, third step 114 can be referred to as processing the coating to form two portions (a first coating portion and a second coating portion). The first coating portion can include the first functionality for bioconjugation, and the second coating portion can include the second functionality to reduce undesired interactions between analytes of interest and the solid support surface. The first coating portion can be formed by immobilizing biomolecules on hydrophilic coating 110. The immobilized biomolecules can be used in sample cleanup, target capturing and digestions steps in workflows used for analysis of biotherapeutics, a protein, or a nucleic acid.

The second functionality can be hydrophilic, and a diol can impart the hydrophilicity. The first functionality can be an aldehyde, and the conjugation of the biomolecules can be via reductive amination. The diol of the second functionality can be used in a controlled process to get the aldehyde of the first functionality.

The conjugation of the biomolecules can be via reductive amination when the first functionality is an aldehyde. When the first functionality is an epoxide, the conjugation of biomolecules can be via the epoxide.

Functionality for bioconjugation 116 can be formed by ring opening or hydrolyzing the hydrophilic surface groups 130. For example, when hydrophilic surface groups 130 are epoxides, the epoxides can be hydrolyzed or ring opened to from diol groups, the diol groups being the functionality for bioconjugation 116.

The ratio of the second functionality to the first functionality can be at least 5%, 15%, 25%, 35%, 45%, 55%, 65%, 75%, 85%, or 95% (any intervening value or range, e.g., at least 12% or between 7% and 24%) of a total surface coverage. And the total surface coverage can be of a thickness to ensure no undesired interactions occur between solid support 112 and an analyte of interest or an undesired compound. For example, the total surface coverage can be greater than 4 μmoles/m², 5 μmoles/m², 9 μmoles/m², or 15 μmoles/m².

Coating 110 can be a hetero-functional coating that is activated by alkoxy silane. The alkoxy silane can be an aldehyde. In some examples, conjugating biomolecules includes conjugating the biomolecules through the aldehyde via reductive amination. The alkoxy silane can also be an acryloxy silane. And the biomolecules can be conjugated through the acryloxy silane via Michael addition. The alkoxy silane can also be a carboxyl silane n-hydroxysuccinimide ester. And the biomolecules can be conjugated through the substitution reaction. That is, the biomolecules can be immobilized by reacting via substitution reaction. When the alkoxy silane is an amine silane, the amine silane can be further reacted with a diacrylate group or a dialdehyde group. The immobilized biomolecules can be reacted with the dialdehyde group via reductive amination. And the immobilized biomolecules can also be reacted with the diacrylate group via Michael addition.

Processing hydrophilic coating 110B to form the second functionality 130 of the second coating portion can be accomplished hydrolyzing the epoxide groups in a controlled manner to convert a fraction of the epoxide groups into surface diols. The fraction of epoxide groups not converted into surface diols can be used for processing the first functionality 116 of the first coating portion, and the first functionality 116 of the first coating portion can conjugate biomolecules, such as enzymes and/or affinity ligands. The fraction of the epoxide groups converted into surface diols can be greater than about 1%, 10%, 20%, 30%, 40%, 50%, 60%, 70%, 80%, 90%, and intervening values such as 55.1%. The fraction of the epoxide groups not converted into surface diols can be used for immobilizing biomolecules, such as enzymes or affinity ligands.

In some examples, processing the coating comprises hydrolyzing unopened epoxides into hydrophilic diols to form the first coating portion and oxidizing a portion of the hydrophilic diols into aldehydes for bio-conjugating biomolecules via reductive amination. Oxidizing a portion of the hydrophilic diols can include controlling the extent of oxidation of the diols to reach a pre-determined portion of oxidation. In some examples, the amount of hydrophilic diols oxidized into aldehyde can be greater than or equal to 1%, 10%, 20%, 30%, 40%, 50%, 60%, 70%, 80%, 90%, and intervening values such as 55.1%.

The functionality for bioconjugation 116 can include different linker chemistries to create coating 110 with linker chemistry. Coating 110 can have mixed surface chemistry. In some examples, coating 110 can be prepared in a multistep process. The first step involves the preparation of the epoxide containing coating 110, followed by hydrolyzing the epoxide into a diol to make a hydrophilic surface of coating 110. Coating 110 is then hydrophilic and can be activated with different linker chemistries. Linker chemistries can carry functionality for conjugating biomolecules, such as an aldehyde or an acryloxy chemistry. Linker chemistry can also be prepared in a multiple step process using several hetero and homo-functional molecules to form the functionality for bioconjugation 116.

In some examples, a coated solid support can be reacted with amine silane, this is then followed with reaction with a diacrylate or dialdehyde. Immobilizing biomolecules can then be carried out through the acrylate group via Michael addition or reductive amination through the aldehyde. Linker chemistries can have hydrophilic or hydrophobic spacers with variable lengths.

The coating can be processed further by hydrolyzing all the epoxides into diol followed by a controlled oxidation of a certain population of the diol into aldehyde. The resulting surface has both hydrophilic diol and the aldehyde groups that are then used to bio-conjugate biomolecules via reductive amination.

In some examples, surface of coating 110 can include silanol on silica surface or hydroxyl groups on a polymer. The diols created on these surfaces are then oxidized in a controlled manner to yield a population of aldehyde groups used for bio-conjugation and some residue diols for eliminating secondary interactions.

A fourth step 120 immobilizes biomolecules on functionality for bioconjugation 116.

For functionality for bioconjugation 116 that do not have immobilized biomolecules, functionality for bioconjugation 116 can be end capped to quench. Quenching includes the introduction of a material that combines with any unused reactants and effectively stops a reaction. The quenching agent should not engage in the reaction in any way other than to combine with one or more reactants. In some examples, the end capping includes amine containing molecules such as ethanol amine and glycine. In some examples, the end capping includes PEGlated diamines, or hetero functional PEGlated molecules with at least one amine group and a hydrophilic end group.

In some examples, the biomolecules are pre-modified before being immobilized. The biomolecules can be pre-modified with an aldehyde or an acrylate chemistry. The biomolecules can also be modified with biotin.

The immobilized biomolecules can be one or more enzymes, i.e., a single enzyme or mixed enzymes. The enzyme can be chosen from a group comprising protease, lipases, phospholipases, ligases, transferases, oxidoreductases, isomerases, hydrolases, or a mixture thereof.

The immobilized biomolecules are a single enzyme or a mixture of enzymes chosen from a group of a protease enzymes. The protease enzymes can be trypsin, Lyc-C, Asp-N, pepsin, Glu-C, or a mixture thereof. The protease enzyme can also be IdeZ or IdeS, and used in characterizing antibodies and antibody drug conjugates. The enzyme can be a glycosidase for O-glycan and N-glycan profiling and mixtures thereof. For glycosidase, example enzymes include, but are not limited, to PNGase F, Endo H, Endo S, and endo-α-N-acetylgalactosaminidase. When the enzyme is from the family of glycosidase, the enzyme can be used for hydrolysis of glucuronides drug conjugates.

Besides enzymes, the immobilized biomolecules can also be one or more affinity ligands. In some examples, the affinity ligand is an immoglobin-binding protein, such as protein A, G, L or a mixture thereof. The affinity ligand can also be an antigen binding, such as an antibody, nanobody, or a mixture thereof. The affinity ligand can also be an aptamers. The affinity ligand can contain avidin and, in some examples, be used with biotinylated protein samples. The avidin containing affinity ligand can be streptavidin, avidin, or neutravidin.

The immobilized biomolecules can include at least one enzyme and/or at least one affinity ligand. Stated another way, the immobilized biomolecules can be at least one enzyme, at least one affinity ligands, or mixtures thereof. For example, the immobilized biomolecules can be two enzymes and one affinity ligand, one enzyme and two affinity ligands, three enzymes and three affinity ligands, and so forth.

Support surface 112 can include silanol on a silica, glass or hybrid inorganic organic surface or hydroxyl groups on a polymer or cellulose surface. Support surface 112 can include surface hydroxyl groups on a polymer or cellulose surface.

In some examples, second functionality 130 of the second coating portion to conjugate biomolecules can be dispersed across a surface of hydrophilic coating 110C and the first coating portion.

Solid surface 102 is precluded from interacting because solid surface 102 is completely covered by hydrophilic coating 110. Solid surface 102 provides a permanent connection with hydrophilic coating 126.

In some examples, not all of the epoxides are converted to diols, and no diols are oxidized to form aldehyde groups. In this way, only a portion of the epoxides are converted to diols. The remaining epoxides (epoxides not converted to diols) serve as the functionality for bioconjugation and the diols serve as the hydrophilic surface group. For example, a coating is prepared by a pre-polymerizing an alkoxy silane containing an epoxide, prepolymer is reacted with the surface on a particle, resin, membrane, or device. The coating is deposited on a solid support preferably particles with some surface area and processed to yield two functionalities. After deposition, epoxide groups are hydrolyzed in a controlled manner to convert a fraction into diols. Surface diols serve as a hydrophilic part of coating and remaining epoxide groups can be used to immobilize any biomolecules such as enzymes or affinity ligands. One important aspect is to control diol to epoxides ratio for better performance.

Similar silanes could be used as a bonding, but the effect would be different due to process coverage limitations. Bonding coverage typically is only able to reach up to 3 μmoles/m². Reduced coverage leaves unreacted underlying surface groups that are detrimental to secondary interactions.

If the coating is too thin, uniform coating cannot be confirmed, and the underlying surface may not be fully covered. The amount of surface coverage can be greater than the bonding coverage, which is typically 3-4 μmoles/m².

In some examples, coating 110A may contain diol groups instead of epoxide groups. Third step 114 can oxidize a portion of the diol groups to form aldehydes. The aldehydes can function as functionality for bioconjugation. The remaining diol groups will function as hydrophilic surface groups 130.

In some examples, the present disclosure of the hetero-functional coating, such as hydrophilic coating 110C, can be used in an apparatus for the analysis of a protein or nucleic acid before processing with a liquid chromatography detection apparatus. The apparatus can include a compartment with an open volume for receiving biomolecules immobilized on support surface 112. The compartment can be a hollow tube with both ends open before immobilized biomolecules material is added. Both ends can be closed after adding the material to allow the apparatus to operate at a high pressure. High pressure includes pressures of greater than 500 psi, greater than 3000 psi, and greater than 10,000 psi as used temporally or statically to disrupt the conformations of analytes.

In some examples, only one end of the compartment is closed to hold the immobilized materials and the other side is left open to add samples. For example, only one side of the compartment can be closed with a frit to hold the immobilized materials but allow fluid to pass freely.

The present disclosure of the hetero-functional coating, such as hydrophilic coating 110° C., can also be used in an apparatus for the analysis of a protein or nucleic acid before liquid chromatography detection apparatus, where the apparatus selected from the group consisting of vial, pipette, PCR tube, micro well plate, channels on a microchip, or glass slide. And the hetero-functional coating of the present disclosure can be applied directly to the apparatus, and support surface 112 can be an interior surface of the apparatus.

As discussed in the disclosure, the immobilized biomolecules are an enzyme or an affinity ligand. The liquid chromatography detection apparatus can be liquid chromatography—mass spectrometry, liquid chromatography—ultraviolet, or liquid chromatography—fluorescence apparatus.

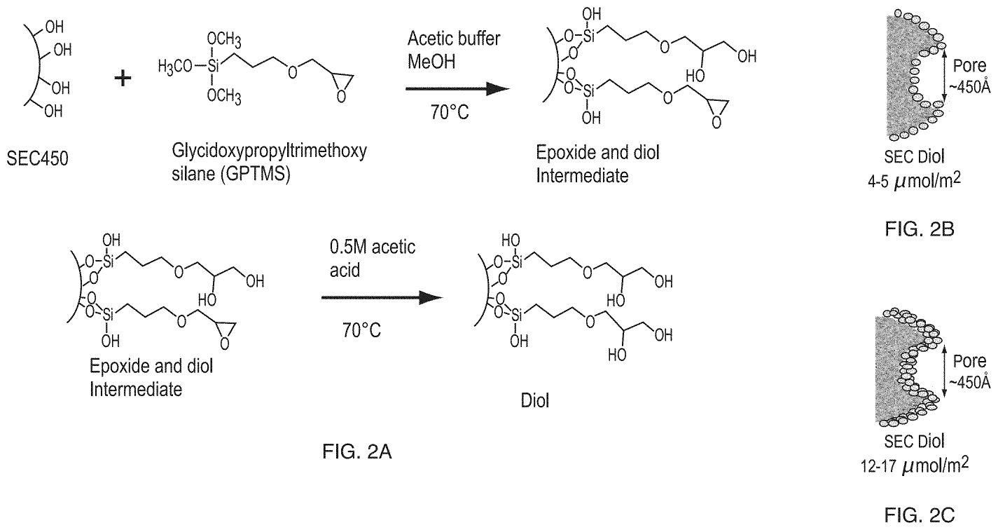

FIG. 2A displays an example of coating on silica particles and surfaces with the diol bonding process. SEC 450 (i.e., an exemplary silica-based solid support) in FIG. 2A is a hybrid-based particle (available from Waters Technologies Corporation, Milford, Mass.). The diol bonding process provides conditions for thicker coatings, e.g., hydrophilic coating 110C. Thicker coatings ensure that the underlying surface, e.g., solid surface 112, is fully covered. For example, by ensuring solid surface 112 is completely covered with hydrophilic coating 110C, no undesired reactions will occur with solid surface 112. In some examples, hydrophilic coating can provide surface coverage of greater than 5 μmoles/m², greater than 9 μmoles/m², or greater than 15 μmoles/m².

FIG. 2B displays an example coating on a particle or surface. FIG. 2C displays an example coating on a particle or surface. In some examples, the diol bonding process can be used for a coating on silica particles and surfaces. For immobilization, conditions can be adjusted for thicker coatings. A thick coating ensures the underlying surface is fully covered. FIG. 2B shows surface coverage of a range of about 4-5 μmoles/m² with pore size of approximately 450 Å. FIG. 2C shows surface coverage of a range of about 12-17 μmoles/m² with pore size of approximately 450 Å.

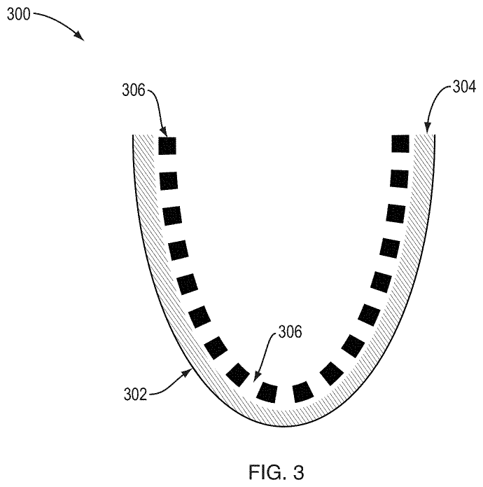

FIG. 3 displays a system 300 with a device 302, a hetero-functional coating 304, and biomolecules 306. Device 302 can include a PCR tube, a microchip channel, a multi-well plate, a tube or vial, a column, and a pipette tip. In some examples, hetero-functional coating 304 immobilizes biomolecules such as affinity ligands or enzymes. In some examples, hetero-functional coating contains a mixture of affinity ligands or enzymes to conjugate the biomolecules. Device 302 can be used to contain biomolecules 306, such as immobilized biomolecules (enzymes on affinity ligands). Device 302 also be coated with hetero-functional coating 304 that is a hydrophilic coating to eliminated undesired interactions with sample. Device 302 can be prepared with immobilized affinity ligands for affinity or enzymes for digestion.

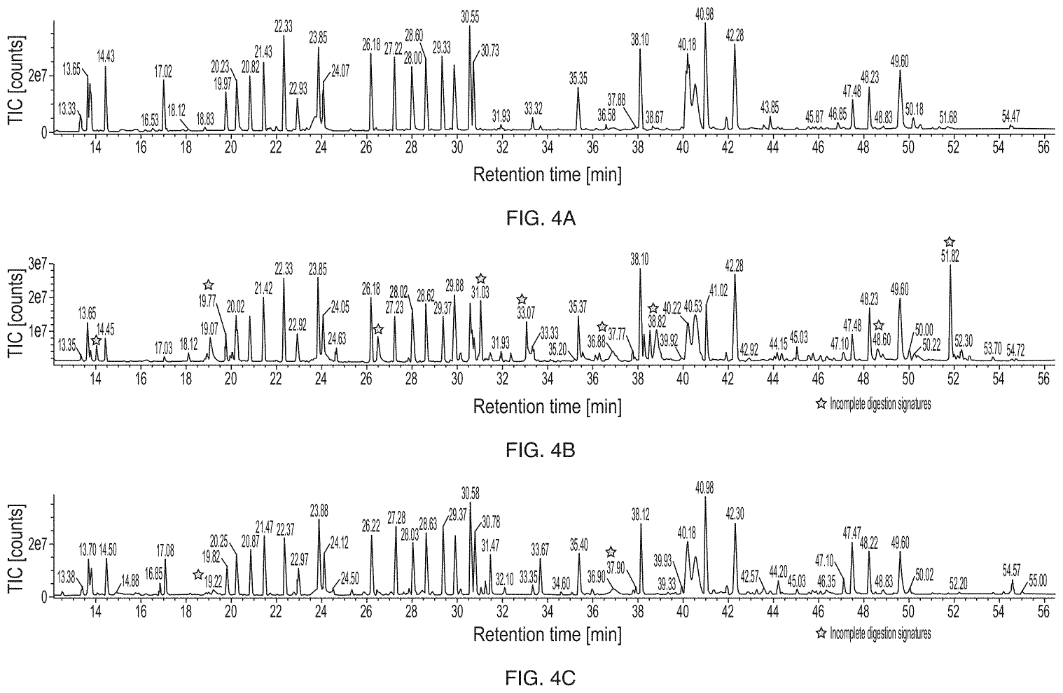

FIGS. 4A, 4B, and 4C display a NIST mAb peptide map for different methods including in-solution enzyme digestion, SMART Digest™, and the present disclosure, respectively. For FIG. 4A, in-solution digestion required two hours at a process temperature of 37° C. As indicated in the map of FIG. 2A, the process yielded only one incomplete digestion signature. For FIG. 4B, SMART Digest™ required ten minutes at a process temperature of 70° C. As indicated in the map of FIG. 2B, the process yielded ten incomplete digestion signatures. For FIG. 4C, a prototype of an example of the present disclosure with immobilized trypsin required ten minutes at a process temperature of 70° C. As indicated in the map of FIG. 2C, the process yielded two incomplete digestion signatures.

Comparing the results of the NIST mAb peptide maps of FIG. 4A, FIGS. 4B, and 4C indicates the advantages of the prototype with immobilized trypsin. In comparison, the prototype of FIG. 4C has superior digestion efficiency than the SMART Digest™ of FIG. 4B. Specifically, the digestion of the prototype of FIG. 4C left two incomplete digestion signatures and the SMART Digest™ of FIG. 4B left ten incomplete digestion signatures. And compared to in-solution digestion of FIG. 4A, the prototype of FIG. 4C has a higher throughput. Specifically, the digestion of the prototype of FIG. 4C required ten minutes while the digestion of the in-solution required two hours.

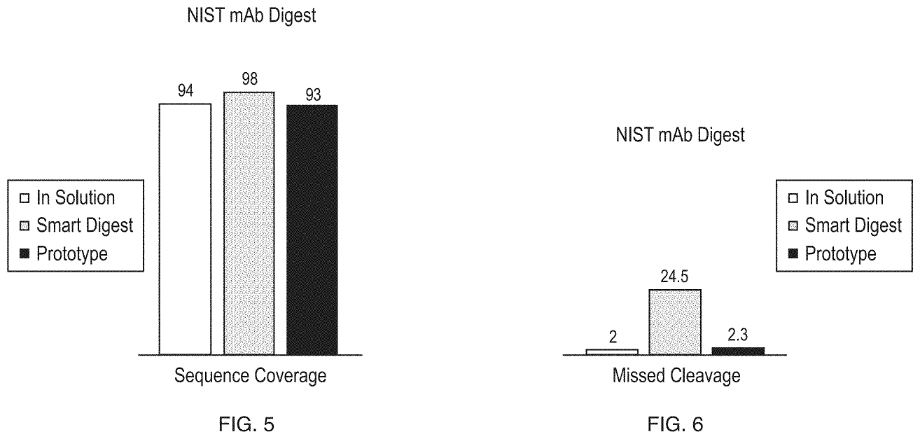

FIG. 5 is a bar graph displaying the calculation of sequence coverage for the peptide maps of FIGS. 4A, 4B, and 4C. The methods including SMART Digest™ (FIG. 4A), in-solution (FIG. 4B) and the prototype of the present disclosure (FIG. 4C) had sequence of 98, 94, and 93, respectively, which are all acceptable levels of sequence coverage. Acceptable levels of sequence coverage have been defined as sequence coverage greater than 90. Variability in sequence coverage values can be attributed to downstream analysis.

FIG. 6 is a bar graph displaying the missed cleavages for in-solution, SMART Digest™, and the prototype with immobilized trypsin. The equation for missed cleavages is the sum of extracted ion chromatograms (XICs) of peptides with missed cleavages divided by the sum of XICs of all identified peptides. The quotient is then multiplied by 100 to calculate the percentage of missed cleavage. See Equation 1.

$$\frac{\sum XICs of peptides with missed cleavages}{\sum XICs of all identified peptides} \times 100 = \% missed cleavage \qquad ( 1 )$$

The SMART Digest™ digestion had 24.5% missed cleavage. While the in-solution digest and the prototype with immobilized trypsin had 2 and 2.3% missed cleavage, respectively. The results show that by changing the digestion method from in-solution to the prototype the time for digestion can be decreased from two hours to ten minutes while maintaining low percentage of missed cleavage of 2.3.

The results show for the same amount of digestion time (ten minutes) the percentage of missed cleavage can be improved from 24.5 to 2.3 when using the prototype of the present disclosure versus SMART Digest™. Improving from 24.5 to 2.3 missed cleavage is a significant improvement. And, the improvement is more significant due to the digestion time being the same. That is, missed cleavage can be improved from 24.5 to 2.3 without having to increase the digest time. The prototype of the present disclosure enables a significant improvement in missed cleavage without having to experience the drawbacks of extending digestion time, i.e., a long digestion time that with the potential negative aspects like autolysis and instability.

Hetero functional coatings have two parts, one part with functionality to attach a biomolecule and a second part contains a hydrophilic group for eliminating secondary interactions. The coating is deposited on the solid support and processed in a controlled manner to yield both functionalities. In the present disclosure, a coating is prepared by a pre-polymerizing an alkoxy silane containing an epoxide, prepolymer is reacted with the surface on a particle, resin, membrane or device. The coating is deposited on a solid support, e.g., particles with some surface area, and processed to yield two functionalities. After deposition, epoxide groups are hydrolyzed in a controlled manner to convert a fraction into diols. Surface diols serve as hydrophilic part of coating and remaining epoxide groups are used to immobilize any biomolecules such as enzymes or affinity ligands. To improve performance, the diol to epoxides ratio is controlled. Some silanes can be used as a bonding, underlying surface groups that are detrimental to secondary interactions. Higher coverage>9 μmole/m² corresponding to thicker coatings lower secondary interactions.

The coating can be processed further, by hydrolyzing all the epoxides into diol followed by a controlled oxidation of a certain population of the diol into aldehyde. The resulting surface has both hydrophilic diol and the aldehyde groups that are then used to bio-conjugate biomolecules via reductive amination. Preferably, silanol on silica surface or hydroxyl groups on a polymer. The diols created on these surfaces are then oxidized in a controlled manner to yield a population of aldehyde groups used for bio-conjugation and some residue diols for eliminating secondary interactions.

Methods of NIST mAb digestion with immobilized enzyme are provided in co-pending application U.S. Ser. No. 17/314,541, is hereby incorporated by reference in its entirety.

### COMPARATIVE EXAMPLES

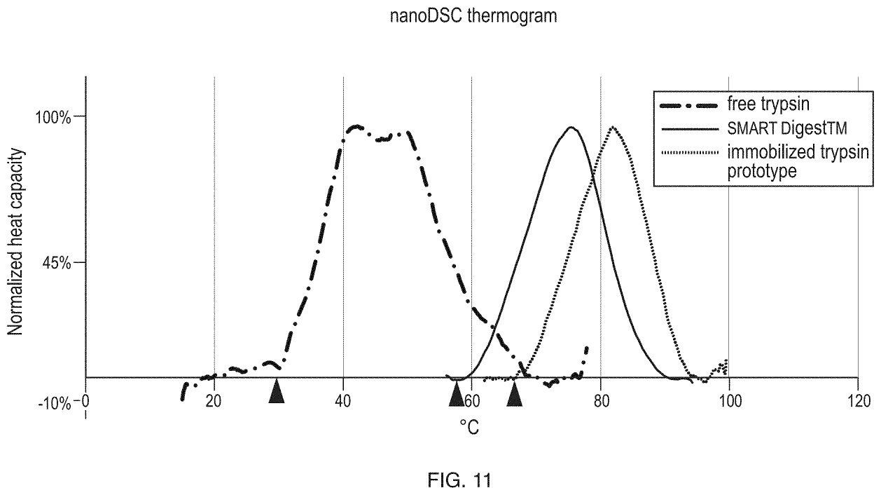

The performance of the present disclosure is compared to SMART Digest™ in FIGS. 11-13. As discussed above, SMART Digest™ (available from Thermo Fisher Scientific, Waltham, Mass.) is another product available for use in digestion. FIG. 11 is a graph of thermograms including a comparison between the present disclosure and SMART Digest™ FIG. 11 displays NanoDSC thermograms of free trypsin, Smart Digest™ trypsin, and one preferred immobilized enzyme (e.g., trypsin) prototype (e.g., silica-based solid support with coating in accordance with the present technology), where Tₘ—temperature where half of the protein is unfolded and Tₒₙₛₑₜ—temperature where protein starts to unfold. For example, the prototype (i.e., silica-based solid support with coating) of the present disclosure with immobilized trypsin showed better thermostability compared to Smart Digest™ (FIG. 1). Tₘ and Tₒₙₛₑₜ for the present disclosure were both greater than Tₘ and Tₒₙₛₑₜ for Smart Digest™ Methods of obtaining thermal stability of immobilized enzyme through NanoDSC are provided in co-pending application U.S. Ser. No. 17/314,541, is hereby incorporated by reference in its entirety.

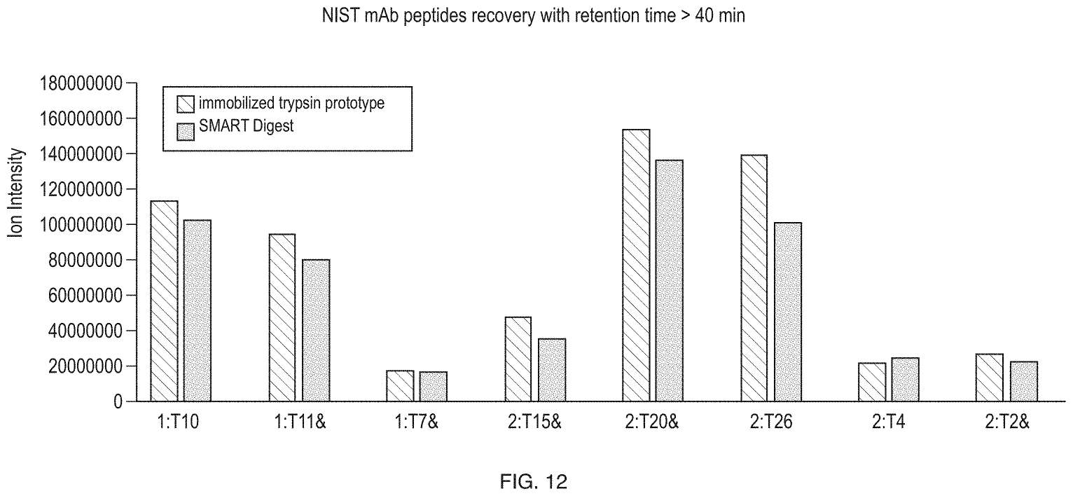

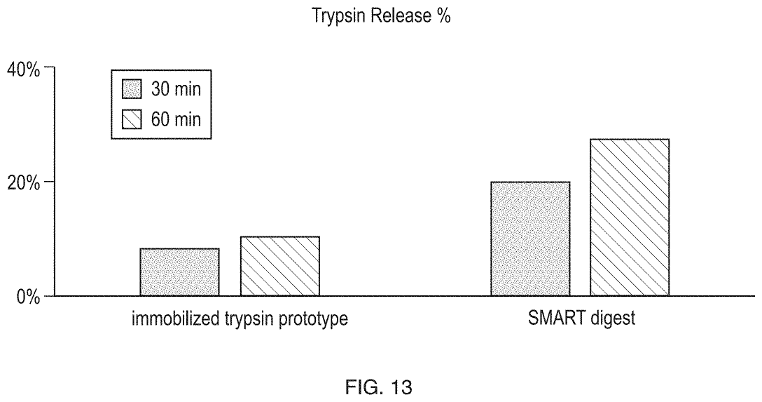

FIG. 12 is a graph of a peptide recovery including a comparison between the present disclosure (i.e., a solid support with coating of the present technology, a prototype) and SMART Digest™. Specifically, FIG. 12 is a graph of a recovery of a mixture of hydrophobic peptides after incubation with immobilized enzymes in accordance with the present disclosure. FIG. 12 demonstrates the recovery % of hydrophobic peptides after mixed with selected immobilized support for only 5 minutes. The prototype of the present disclosure, the modified silica with trypsin, outperformed SMART Digest™ for every enzyme. That is, the prototype in FIG. 12 (i.e., a silica-based solid support with coating) had a greater recovery % for each enzyme than SMART Digest™. Methods for quantification of released trypsin are provided in co-pending application U.S. Ser. No. 17/314,541, is hereby incorporated by reference in its entirety.

FIG. 13 is a graph of released trypsin including a comparison between the present disclosure and SMART Digest™. FIG. 13 is graph of released trypsin after incubated in digestion buffer at 70° C. after 30 minutes and 60 minutes comparing a prototype of the present disclosure (i.e., a silica-based solid support with coating of the present technology) versus SMART Digest™. A preferred product has minimal leakage of the enzyme during the digestion. The prototype of the present disclosure substantially outperformed SMART Digest™ at 30 minutes and 60 minutes. Methods for NIST mAb digestion with immobilized enzyme are provided in co-pending application U.S. Ser. No. 17/314,541, is hereby incorporated by reference in its entirety.

### EXAMPLES

Materials were used as received unless otherwise noted.

Characterization:

Both percent carbon (% C or % carbon) and percent nitrogen (% N or % Nitrogen) values were measured by combustion analysis using a LECO TruMac carbon-nitrogen/sulfur Analyzer (available from Leco Corporation, Michigan, US). The specific surface areas (SSA), and the average pore diameters (APD) of these materials were measured using the multi-point N2 sorption method (Micromeritics ASAP 2400; Micromeritics Instruments Inc., Norcross, Ga.). The SSA was calculated using the Brunauer-Emmett-Teller (BET) method, APD was calculated from the isotherm's desorption leg using the Barrett, Joyner, and Halenda (BJH) method. Particle sizes were measured using a Beckman Coulter Multisizer 3 analyzer (30 μm aperture, 70,000 counts; available from Beckman Coulter Inc., Brea, Calif.). Particle morphology was imaged with Hitachi SEM 53400 (available form Hitachi, Ltd., Tokyo, Japan). The particle diameter was measured as the 50% cumulative diameter of the volume size distribution. Measurements of pH were made with an Oakton pH100 Series meter (available from Cole-Palmer, Vernon Hills, Ill.) and calibrated using ORION® buffers (available from Thermo Electron Corp., Beverly, Mass.) pH buffered standards at ambient tempera-ture immediately before use. Titrations were performed using a METROHM® 716 DMS TITRINO® autotitrator (available from Metrohm AG, Hersau, Switzerland), and are reported as milliequivalents per gram (mequiv/g). Coverage levels for the epoxide were determined by titrating the OH-liberated upon addition of sodium thiosulfate.

### Example 1

Preparation of a Thick Hetero-Functional Coating on Hybrid Organic Porous Silica

The reaction buffer preparation step is important for the final product's coating efficiency. This reaction is pH sensitive with pH values between 5.50 and 5.55, resulting in reproducible thick hetero functional coatings, multiple layers coating, and pH 4.00-5.00 favoring thin coating or a monolayer. Increasing pH above 5.55 risks possible gelation. 20 mM buffer pH 5.54±0.01 was prepared by dissolving 2.379 g of sodium acetate trihydrate (available from Fisher Scientific, Waltham, Mass.) in 1000 mL of MilliQ water and adjusting pH with glacial acetic acid (J. T. Baker Inc., Philipsburg, N.J.).

Hetero-functional coating of hybrid porous silica particles was prepared using a freshly made 20 mM pH 5.54 sodium acetate. To clean 3-neck 250 mL round bottom flasks equipped with a mechanical stirrer, and an appropriate blade, a condenser, a temperature-controlled heating, a temperature probe, and an adapter, 100 mL of the reaction buffer was added. The buffer solution was pre-heated to 70° C. while stirring, the pH was monitored at the set temperature and remained constant at pH 5.54. To a clean 50 mL beaker, 6.50 g of redistilled (3-glycidoxypropyl) trimethoxysilane (GPTMS) (available from Gelest, Inc., Morrisville, Pa.) was pre-dissolved in 3.12 mL of methanol using a vortex mixer for 1 minute and sonicated in a water bath for 1 minute at room temperature, then transferred with mixing into the flask containing the hot buffer. The reaction mixture was incubated with continuous stirring at 70° C. for 60 minutes. Reaction pH was measured after 55 minutes at 70° C. During the 60-minute hold in hot pH 5.4 buffer, GPTMS undergoes pre-polymerization resulting in a pH change. In this case, the pH changed to 6.42. If the pre polymerized GTPMS pH after the incubation period rises above 6.5, the reaction is stopped due to possible gelation; sometimes, the solution may turn turbid.

On the other hand, if the pH does not change from the original buffer pH 5.5, the reaction should also be stopped, indicating insufficient pre-polymerization. At precisely 60 minutes of incubation, a total of 10 grams of hybrid organic porous silica materials (surface area 79 m²/g, APD 450 Å, Waters Corporation, Milford, Mass.) was added to the hot reaction mixture and incubation continued at 70° C. for 20 hours, after which the reaction mixture was then cooled down to 40° C. After cooling to 40° C., particles were washed three times with 100 mL MilliQ water and two times with 100 mL 2-propanol, dried under a constant stream of nitrogen for at least 20 minutes, and stored at 4° C. A small sample of the final material was submitted for analysis. Carbon content was measured by combustion analysis and coating thickness calculated from the difference between % C before and after coating. Titration was used to quantify epoxy groups. The resulting Product 1 coating thickness was 15 μmol/m² with 61% of residue epoxide groups and 31% diol groups.

### Example 2

Trypsin Bioconjugation on Thick Coated Hetero-Functional Hybrid Organic Porous Silica Through Epoxide Linker Chemistry

Trypsin bioconjugation on hetero-functional coated hybrid organic porous silica particles was performed by salting-out precipitation with a high concentration ammonium sulfate solution in phosphate buffer pH 8. The following chemicals were added into a clean 100 mL volumetric flask: 153 mg of dibasic dihydro sodium phosphate (available from MilliporeSigma, St. Louis, Mo.), 16.7 mg of monobasic sodium phosphate (available from MilliporeSigma, St. Louis, Mo.), and 38.06 g of ammonium sulfate (available from MilliporeSigma, St. Louis, Mo.). Up to 50 mL of MilliQ water was added to the flask and sonicated in a bath sonicator until all chemicals were thoroughly mixed. The flask was then filled to 100 mL with MilliQ water, and pH was adjusted to pH 8 with 10 M NaOH. The final concentrations were 2.88M ammonium sulfate, and 10 mM phosphate buffer pH 8 and are important for salting out. To a clean vial, 18 mg of L-(tosylamido-2-phenyl) ethyl chloromethyl ketone (TPCK) treated Trypsin (available from Worthington Biochemical Corp., Lakewood N.J.) and 70 mg of trypsin inhibitor, Benzamidine available from MilliporeSigma, St. Louis, Mo.) was added and mixed with 1.168 mL of 10% glycerol (available from MilliporeSigma, St. Louis, Mo.)/MilliQ water (g/g) and sonicated in a bath sonicator at room temperature for 1 minute. 1.168 mL of the immobilization buffer (2.88M ammonium sulfate, 10 mM phosphate buffer pH 8) was added dropwise to trypsin solution with vortex mixing. To a clean glass vial, 300 mg hetero-functional coated hybrid organic porous silica particles (Example 1, Product 1, Waters Corporation, Milford, Mass.) were dispersed in 10 mL of immobilization buffer (2.88M ammonium sulfate, 10 mM phosphate buffer pH 8) and sonicated in a bath sonicator for 1 minute at room temperature to disperse the particles. Trypsin solution was added slowly, dropwise to the particles solution with vortex mixing at room temperature. Once trypsin solution addition was completed, the reaction was put on an inversion mixer (RotoBot rotation mixer available from Benchmark Scientific Inc., Edison, N.J.) and incubated for 20 hours at room temperature with inversion mixing. Controlling the mixing minimizes foaming, which can result in lower bioconjugation efficiency. After 20 hours, 122 mg of glycine (available from MilliporeSigma, St. Louis, Mo.) was added and left mixing for 30 more minutes to quench the residual epoxide group. The reaction vial content was transferred into 50 mL centrifuge tubes (available from Corning, Sigma Aldrich, St. Louis, Mo.) for washing. The particles were washed twice with 40 mL pH 4 water (prepared by adding drops of hydrochloric acid to MilliQ water) by soaking the particles for 15 minutes, followed by centrifugation at 4000 rpm for 10 minutes, washed four times with 40 mL MilliQ water with centrifugation. Finally, the material was redispersed in a storage solution containing 10 mM CaCl₂ in 0.01% formic acid and stored at 4° C. A small sample of the final material was analyzed. The amount of bioconjugated Trypsin was quantified using micro BCA™ assay kit (available from Thermo Fisher Scientific, Waltham, Mass.). The final Product 2 had a trypsin content of 34.4 mg/g of particles corresponding to 57% bioconjugation efficiency. n

### Example 3

Epoxide Group Hydrolysis or Ring Opening into Diols

Approximately 5 g of diol/epoxide Product 1 from Example 1 (15 mole/m² coating thickness, 61% residue epoxide) was added in a clean 100 mL 3 neck round bottom flask equipped with a stirring shaft, bearing and appropriate blade, a water-cooled condenser, a temperature-controlled heating mantle, a temperature probe, and an adapter. To the flask, 50 mL of 1 M acetic acid solution and the reaction mixture incubated with mixing at 70° C. for 20 hours followed by cooling down. After cooling below 40° C., the particles were washed with an excessive amount of MilliQ water until the pH>5, followed by washing two times with 50 mL methanol and dried in a vacuum oven at 70° C. for 16 hours. A sample of the final material was analyzed. The final material, Product 3, had 0.0 μeq/g residue epoxide, a confirmation of complete hydrolysis by epoxide titration.

### Example 4

Controlled Diol Groups Oxidation to Aldehyde for Bioconjugation

The hydrophilic diol-coated hybrid porous silica particles of Product 3 of Example 3, were oxidized in a controlled manner using sodium periodate (available from MilliporeSigma, St. Louis, Mo.). The oxidant solution was prepared in an amber glass bottle immediately before use to protect from light-induced decomposition. To a clean 100 mL three-neck clear glass flask equipped with a stirring shaft, bearing, and appropriate blade, 4.5 g of Product 3 of Example 3 and 70 mL of a 0.1M sodium periodate solution were mixed for 5 hours at 25° C. The level of oxidation is controlled by the concentration, higher concentrations resulting in a higher diol oxidation level into aldehydes. Upon reaction completion, the product was washed three times with 10 mL/g MilliQ water and washed two times with 10 mL/g ethanol. The resulting Product 4 was dried under a positive pressure stream of nitrogen for 20 minutes and stored at 4° C. The aldehyde materials were characterized with fourier-transform infrared spectroscopy (FTIR); carbon content was measured by combustion analysis. Aldehyde groups were quantified by reductive amination with a primary amine molecule such as ethanolamine. To a 50 mL centrifuge tube (available from Corning, Fisher Scientific, Waltham, Mass.), 1.0 g of Product 4 from Example 4 was added and dispersed in 10 mL of 100 mM ethanolamine pH 9.5 (pH of ethanolamine solution was adjusted with 1N hydrochloric acid). The reaction mixture was left mixing on a vortex for 10 minutes at room temperature. To a clean vial, 760 mg of sodium borohydride (available from MilliporeSigma, St Louis, Mo.) was added and pre-mixed in 3 mL of 100 mM ethanolamine pH 9.5. The sodium borohydride solution was added to the reaction mixture, covered with a parafilm and left mixing on a vortex for 3 hours at room temperature. Afterward, the materials were washed with a copious amount of water until the wash had a pH<6. The materials were then washed four times with 10 mL/g of methanol and dried in a vacuum oven for 16 hours in 80° C. A sample of the final material was analyzed. Carbon and nitrogen content was measured by combustion analysis, and coverage was calculated for the difference between % C or % N before and after reductive amination. Final material % N calculation resulted in 5.4 mole/m² of ethanolamine, corresponding to the same amount of aldehyde groups present after the oxidation step, and 36% conversion of diol to the aldehyde of the Product 4.

### Example 5

### Trypsin Bioconjugation on Thick Coated Hetero-Functional Coated Aldehyde Activated Hybrid Organic Porous Silica Particles

TCPK treated Trypsin was bioconjugated on hetero-functional hybrid organic porous silica particles Product 4 by reductive amination. The reaction was carried out in 100 mM triethanolamine (available from Sigma Aldrich, St Louis, Mo.) pH 9 buffer, 10 mM calcium chloride (available from Sigma Aldrich, St Louis, Mo.), and 10% (g/g) glycerol (available from Sigma Aldrich, St Louis, Mo.) as a dispersant. To a clean 20 mL vial, 12 mg of TPCK treated Trypsin (available from Worthington Biochemical Corporation, Lakewood, N.J.) and 46.8 mg of benzamidine (available from Sigma Aldrich, St Louis, Mo.) were pre-mixed in 10 mL of the buffer by sonicating for 1 minute. The trypsin mixture was added to a clean 200 mL flask, and an extra 40 mL of the pH 9 buffer was added and mixing continued for 10 minutes at room temperature. The mixing speed is controlled to prevent foam formation, which can significantly impact bioconjugation efficiency. Approximately, 300 mg of hetero-functional coated particles, Product 4, was pre-mixed with 40 mL of the bioconjugation buffer, sonicated in a bath sonicator for 1 minute and transferred with mixing into the flask with Trypsin. The mixture was allowed to mix for 10 minutes. To a clean scintillation vial, 400 mg of sodium cyanoborohydride (available from Sigma Aldrich, St Louis, Mo.) reducing agent was pre-dissolved in 5 mL bioconjugation buffer and added to the flask containing Trypsin and the particles. The bioconjugation reaction was carried out for 3 hours at room temperature. After 3 hours of mixing, the materials were transferred into a 600 mL centrifuge tube, washed once with an excess of pH 4 MilliQ water (prepared by adding a few drops of hydrochloric acid), and centrifuged at 4000 rpm for 10 minutes. Unreacted aldehyde groups were quenched by redispersing the materials in 1M ethanolamine solution at pH 9.5, with 10 mM CaCl₂, 46.8 mg of benzamidine (available from Sigma Aldrich, St Louis, Mo.), and 400 mg of cyanoborohydride. The reaction mixture was left mixing at room temperature for 30 minutes. The materials were then washed twice with 45 mL pH 4 MilliQ water (prepared by adding a few drops of hydrochloric acid) with soaking for 15 minutes for each wash and centrifuging at 4000 rpm for 10 minutes, and washed four times with MilliQ water. After washing, the materials were redispersed in 0.01% Formic acid and 10 mM CaCl₂ yielding Product 5 stored at 4° C. A sample of Product 5 was submitted to be analyzed. The amount of bioconjugated Trypsin was quantified with BCA™ assay (available from Thermo Fisher, Waltham, Mass.). Product 5 had a trypsin content of 38 mg/g particles corresponding to 96% immobilization efficiency.

### Example 6

Preparation of Thin Hetero-Functional Coating on Hybrid Organic Porous Silica Particle

To prepare low pH buffer, 0.412 g of sodium acetate trihydrate (available Fisher Scientific, Waltham, Mass.) was dissolved in 1000 mL of MilliQ water and pH adjusted with glacial acetic acid (available from J. T. Baker Inc., Philipsburg, N.J.) to pH 4.0. To a clean round bottom flask, equipped with a stirring shaft, appropriate blade, condenser, a temperature-controlled heating, a temperature probe and an adapter, 60 mL of the pH 4 was added. The buffer was preheated to 70° C. with mixing for 1 hour, and pH was measured. Reaction buffer pH changed slightly from pH 4.01 to 3.95. To a clean 20 mL vial, 2.130 g of redistilled (3-Glycidoxypropyl) trimethoxysilane (GPTMS) (available from Gelest Inc., Morrisville, Pa.) was added and premixed on vortex mixer with 0.530 mL of methanol, sonicated for 1 minute, transferred with mixing into the flask with hot buffer and continued incubation at 70° C. for 55 minutes. After 55 minutes of heating, the pH of the mixture was recorded as 4.03. At precisely 60 minutes of incubation, of the hybrid organic porous silica particle (SSA 79 m²/g, APV 450 Å, Waters Corporation, Milford, Mass.) was added to the hot reaction mixture and continued mixing at 70° C. for 20 hours, which was followed by cooling down. After cooling to less than 40° C., the materials were washed three times with 100 mL MilliQ water and two times with 2-propanol, dried under constant nitrogen stream for at least 20 minutes, yielding Product 6 stored at 4° C. A sample of Product 6 was analyzed. Carbon content was measured by combustion analysis, and the difference in % C before and after coating was used to calculate coating thickness. The residue epoxy group content was quantified by the epoxy group titration method. Product 6 has a coating thickness of 3.13 mole/m² with 37% of residual epoxide groups and 67% diol groups. The coating thickness was controlled by changing the silane concentration, the pH between 4-5, and the silane charge.

### Example 7

### Trypsin Bioconjugation on Thin Hetero-Functional Coated Hybrid Organic Porous Silica Particles with Epoxide Group

Trypsin immobilization on thin hetero-functional coated hybrid organic porous silica particles of Product 6 of Example 6 was performed by the same salting-out method with 2.88 M ammonium sulfate solution in 10 mM phosphate pH 8 buffer, as described for Product 2 of Example 2. To a clean vial, 18 mg of TPCK treated Trypsin (available from Worthington Biochemical Corp., Lakewood, N.J.) and 70 mg of trypsin inhibitor, Benzamidine (available from Sigma Aldrich, St. Louis, Mo.) and mixed with 1.168 mL of 10% glycerol (available from Sigma Aldrich, St. Louis, Mo.)/MilliQ water (g/g) and sonicated in a bath for 1 minute. Additional 1.168 mL immobilization buffer was added dropwise to trypsin solution with vortex mixing. To a clean glass vial, 300 mg Product 6 was dispersed in 10 mL of immobilization buffer and sonicated for 1 minute at room temperature. Trypsin solution was added dropwise to the particles solution with vortex mixing at room temperature to precipitate the Trypsin on the surface of the particles. Once all the Trypsin solution had been added, the reaction was put on an inversion mixer (RotoBot rotation mixer available from Benchmark Scientific, Edison, N.J.) and left for 20 hours at room temperature for the completion of the reaction. After 20 hours, 122 mg of glycine (available from MilliporeSigma, St. Louis, Mo.) was added to the reaction mixture and left mixing for 30 more minutes to quench the residual epoxide group. The vial content was transferred to 50 mL centrifuge tubes (available from Corning, Sigma Aldrich, St. Louis Mo.), washed twice with 40 mL pH 4 water (prepared by adding drops of hydrochloric acid to MilliQ water) by soaking for 15 minutes in between washes, followed by centrifugation at 4000 rpm, washed again four times with MilliQ water. The final material, Product 7, was redispersed in a storage solution containing 10 mM CaCl₂ in 0.01% Formic acid and stored at 4° C. The amount of bioconjugated Trypsin was quantified using micro BCA™ assay kit (available from Thermo Fisher, Waltham, Mass.). Product 7 had a trypsin content of 30.3 mg/g, corresponding to 50.6% immobilization efficiency.

### Example 8

Preparation of Thin Hydrophilic Diol Coated Hybrid Organic Porous Silica Particles

Reaction buffer was prepared as described in Example 1 with one exception, buffer pH was adjusted to 5. To a clean three-neck 1000 mL round bottom flask equipped with a stirring shaft, appropriate blade, condenser, heating, 720 mL of the pH 5 buffer was added. The buffer was preheated to 70° C. with mixing for 1 hour, and with pH monitoring. To a clean 100 mL three-neck round bottom flask, 62.8 g of redistilled (3-Glycidoxypropyl) trimethoxysilane (GPTMS) (available from Gelest, Inc., Morrisville, Pa.) premixed with 14.7 mL of methanol, sonicated for 2 minutes, transferred with mixing into the flask with hot buffer and continued incubation at 70° C. for 55 minutes. After 55 minutes of heating, the pH of the mixture was recorded as 5.2. At precisely 60 minutes of incubation, 150 g of the porous hybrid organic porous silica (SSA 77 m²/g, APD 450 Å, Waters Corporation, Milford, Mass.) was added to the hot reaction mixture and continued mixing at 70° C. for 20 hours, which was followed by cooling down. After cooling to less than 40° C., the materials were washed three times with 100 mL MilliQ water and two times with 2-propanol, and dried under the constant nitrogen stream for at least 20 minutes. The materials were hydrolyzed immediately after drying, following the procedure described in Example 3. To a clean three-neck 1 L round bottom flask, the freshly prepared materials were added and mixed with 750 mL 1M acetic acid solution prepared in Example 3. The reaction mixture was incubated with mixing at 70° C. for 20 hours followed by cooling down. After cooling below 40° C., the materials were washed with an excessive amount of MilliQ water until the pH>5, followed by washing two times with 50 mL methanol and dried in a vacuum oven at 70° C. for 16 hours to yield Product 8. Carbon content was measured by combustion analysis, and complete hydrolysis was confirmed with epoxide titration. The final Product 8 had 0.0 μeq/g residue epoxide and a coating thickness of 4.6 μmol/m² calculated from the change in % C before and after the coating process.

### Example 9

Aldehyde Activation of Thin Hydrophilic Coated Hybrid Organic Porous Silica Particles

Oxidation of the thin hydrophilic coated hybrid organic porous silica particles of Product 8 of Example 8 was completed using the procedure described in Example 4 to yield Product 9. Similarly, aldehyde materials of Product 9 were characterized with FTIR. Carbon content was measured by combustion analysis, particle morphology was confirmed with SEM, particle sizes were measured using a Beckman Coulter Multisizer 3 analyzer (30 μm aperture, 70,000 counts; available from Beckman Coulter Inc., Brea, Calif.), and aldehyde group coverage was quantified using reductive amination as described in Example 4. Final material % N was measured with combustion and used to calculate the amount of ethanol amine which corresponds to the amount of aldehyde on Product 9. Based on these calculations, Product 9 had 2.7 mole/m² aldehyde groups after oxidation step corresponding to 59% diol to aldehyde conversion.

### Example 10

Trypsin Bioconjugation with Aldehyde Activated Thin Hetero-Functional Coated Hybrid Porous Particles

TCPK treated Trypsin was bio conjugated on hetero-functional coated oxidized hybrid porous material, Product 9, by reductive amination through the aldehyde groups following the procedure described in Example 5. The amount of Trypsin bioconjugated was quantified with Fisher micro BCA™ assay (available from Thermo Fisher, Waltham, Mass.). The final product, Product 10, had 36.5 mg/g particles' trypsin content corresponding to 91% immobilization efficiency.

### Example 11

Preparation of Thick Hetero-Functional Coating on Silica Core-Shell Particles

The hetero-functional coated silica core-shell particle was prepared following the procedure described in Example 1. Materials of different coating thickness were achieved by changing GPTMS concentration and particle concentration while using the same buffer pH. 20 mM pH 5.5 sodium acetate buffer was prepared and used immediately. In these reactions, 100 mL of the reaction buffer was added to a clean three-neck 200 mL round bottom flask equipped with a stirring shaft, bearing and an appropriate blade, a water-cooled condenser, temperature-controlled heating mantle, a temperature probe, and an adapter. The reaction buffer was preheated to 70° C. while stirring. Redistilled (3-glycidoxypropyl)trimethoxysilane (GPTMS) (available from Gelest, Inc., Morrisville, Pa.) premixed with 21% V/V methanol on a vortex mixer, sonicated for 1 minute, then transferred with mixing into the flask containing hot buffer with continued incubation at 70° C. for 60 minutes. The amount of GPTMS was varied to yield different products; see Table 1 below. pH was monitored after 55 minutes of incubation and before the addition of solid materials. At exactly 60 minutes of incubation, different amounts of the silica core-shell materials (SSA 23 m²/g, APD 450 Å, available from Waters Corporation, Milford, Mass.) were added to the flasks with hot buffer to yield products varying coating thickness as shown in Table. The reaction mixtures were incubated at 70° C. for 20 hours washed and dried, similar to Example 1 above. These materials were hydrolyzed immediately after drying to the following procedure described in Example 3.

TABLE 1
Hetero-functional coated silica core-shell particles

| | GTMS | Amount of | |
|---|---|---|---|
| | Con- | GPTMS added | Coating |
| | centration | per unit SSA | thickness |
| Product | (M) | (μmol/m²) | (μmol/m²) |
| 11A | 0.06 | 35 | 3.9 |
| 11B | 0.11 | 56 | 5.5 |
| 11C | 0.18 | 57 | 12.6 |
| 11D | 0.18 | 94 | 27 |

### Example 12

Controlled Aldehyde Activation on Hydrophilic Coated Silica Core-Shell Particles

Oxidation of hydrophilic coated silica core-shell particles of Product 11C was achieved by using the procedure similar to Example 4 to yield Product 12. A sample of the final materials were characterized with FTIR; carbon content was measured by combustion analysis, particle morphology was confirmed with SEM, particle size with Beckman Coulter Multisizer 3 analyzer (30 μm aperture, 70,000 counts; available from Beckman Coulter Inc., Brea, Calif.), and thermogravimetric analysis. Aldehyde group coverage was quantified using reductive amination reaction with ethanolamine as described in Example 4. Final material % N was used to calculate the amount of aldehyde. Product 12 had 4.7 mole/m² aldehyde groups after oxidation step corresponding to 37% diol to aldehyde conversion.

### Example 13

Trypsin Bioconjugation on Thick Hetero-Functional Coated Silica Core-Shell Particles

Trypsin was immobilized on materials of example 12 Product 12 following procedure described in Example 5 and stored at 4° C. yielding Product 13. The trypsin amount immobilized on these materials was 18 mg/g particles with 91% immobilization efficiency.

### Example 14

Controlling Thickness of Hetero-Functional Coating on Hybrid Organic Porous Silica Particles

The hetero-functional coating thickness can be controlled by several factors such as the GPTMS concentrations, the ratio of the total surface area to the amount of silane added, and pH. Several products were prepared following Example 1, using hybrid porous materials (SSA 85 m²/g, APD 300 Å, available from Waters Corporation, Milford, Mass.), a variable amount of GPTMS, and different buffer pH prepared following Example 1. All these materials were hydrolyzed following the procedure described in Example 3. See Table 2 below for materials with different hydrophilic diol coating thicknesses.

TABLE 2
Controlling hetero-functional coating thickness
on hybrid organic porous silica particles

| | GTMS | GPTMS | | | |
|---|---|---|---|---|---|
| | Con- | silane per | | Coating | |
| | centration | unit SSA | | thickness | |
| Product | (M) | (μmol/m2) | pH | (μmol/m2) | Comments |
| 14A | 0.18 | 21 | 5.54 | 9.3 | Different |
| 14B | 0.16 | 20 | 5.59 | 8.0 | silane |
| | | | | | concentration |
| 14C | 0.28 | 22 | 5.5 | 5.4 | Particle |
| 14D | 0.28 | 33 | 5.54 | 8.3 | concentration |
| 14E | 0.28 | 39 | 5.54 | 17 | |
| 14F | 0.23 | 22 | 5.5 | 7.4 | |
| 14G | 0.23 | 33 | 5.5 | 11 | |
| 14H | 0.23 | 66 | 5.0 | 4.2 | pH |
| 14I | 0.23 | 66 | 5.4 | 16 | |
| 14J | 0.23 | 66 | 5.5 | 19.2 | |
| 14K | 0.23 | 66 | 5.6 | 22 | |

### Example 15

Hetero-functional coating thickness may also be prepared with materials of different porosity. Several products were prepared by varying the concentration of GPTMS in the reaction mixture, the ratio of the total surface area to the amount of silane added, pH, and the amount of methanol used to pre-dissolve the silane before the reaction. Methanol aids in the solubility of silicate species formed during the reaction. Higher amounts of methanol result in thinner coatings. The products were achieved by following the procedure described in Example 1, using hybrid porous materials (SSA 79 m2/g, APD 450 Å, available from Waters Corporation, Milford, Mass.). See Table 3 below for materials with different hydrophilic coating thicknesses. All materials were hydrolyzed to ring open the epoxide following the procedure in Example 3.

TABLE 3
Impact of methanol on hetero-functional coating
thickness on hybrid organic porous silica particles

| | GTMS | Silane | | | |
|---|---|---|---|---|---|
| | Con- | per | | | Coating |
| | centration | SSA | | V/V % | thickness |
| Product | (M) | (μmol/m²) | pH | Methanol | (μmol/m²) |
| 15A | 0.15 | 23 | 4.0 | 17 | 3 |
| 15B | 0.12 | 35 | 5.5 | 17 | 6 |
| 15C | 0.23 | 35 | 5.54 | 17 | 13 |
| 15D | 0.26 | 38 | 5.54 | 17 | 15 |
| 15E | 0.23 | 33 | 5.54 | 17 | 21 |
| 15F | 0.27 | 34 | 5.54 | 21 | 13 |
| 15G | 0.23 | 50 | 5.54 | 21 | 22 |
| 15H | 0.23 | 50 | 5.54 | 35 | 19 |
| 15I | 0.23 | 50 | 5.54 | 45 | 15 |

### Example 16

Controlled Oxidation of Hydrophilic Diol Coated Hybrid Organic Porous Silica Particles of Constant Coating Thickness

Selected materials from Example 15, Product 15C and Product 15F both with coating coverage of 13 μmol/m², were used for controlled diol oxidation using the modified procedure described in Example 4. Different materials were exposed to the oxidant solution to yield different oxidation levels, resulting in different diol/aldehyde ratios. See Table 4 below.

TABLE 4
Hetero-functional coated hybrid organic porous
silica materials with different aldehyde coverages

| | Oxidant | | | |
|---|---|---|---|---|
| | Con- | | Aldehyde | |
| | centration | Time | coverage | % con- |
| Product | (M) | (h) | (μmole/m²) | version |
| 16A | 0.1 | 0.5-5 | 4.72 | 36 |
| 16B | 0.03 | 0.5-5 | 3.09 | 24 |
| 16C | 0.02 | 0.5-5 | 2.5 | 19 |
| 16D | 0.01 | 0.5-5 | 1.2 | 9 |
| 16E | 0.008 | 0.5-5 | 0.7 | 5 |

### Example 17

Controlled Oxidation of Hydrophilic Diol Coated Hybrid Organic Porous Silica Particles for Different Coating Thickness

Selected materials with different coating thickness were exposed to same concentration of oxidant as described in Example 4, yielding products of varying oxidation levels and similar % diol conversion. See Table 5 below.

TABLE 5
Controlled oxidation of hydrophilic diol coated
hybrid organic porous silica particles

| | Coating | Aldehyde | |
|---|---|---|---|
| | thickness | coverage | % Diol |
| Product | (μmol/m²) | (μmole/m²) | conversion |
| 17A | 3.91 | 1.27 | 33 |
| 17B | 7.64 | 3 | 39 |
| 17C | 12.5 | 4.7 | 37 |
| 17D | 12.7 | 4.42 | 35 |
| 17E | 17.3 | 8.1 | 47 |
| 17F | 19.44 | 8 | 41 |
| 17G | 21.51 | 9.5 | 44 |

### Example 18

NIST mAb Digestion with Trypsin Immobilized on Hetero-Functional Coated Hybrid Organic Porous Silica

NIST mAb was used as a model protein to evaluate prototypes' performance prepared by immobilizing trypsin on a hetero-functional coated hybrid organic porous silica using the procedure described in Example 5 above. Approximately 50 pg of NIST mAb was denatured and reduced in 8 M guanidine buffer with 5 mM Dithiothreitol (DTT) for one hour followed with alkylation for 30 minutes in the dark with 15 mM Iodoacetamide (IAM). The alkylated protein was then desalted using NAP-5 columns (available from GE Healthcare, General Electric, Boston, Mass.) and mixed with 15 pL immobilized trypsin. For each of the products, 15 pL of immobilized trypsin slurry; digested at 70° C. for 10 minutes on a shaker, after which the sample was centrifuged and 100 pL of supernatant was submitted for LC-MS analysis. The peptide map generated through a standard LC-MS assay from different prototypes had a similar profile to in-solution trypsin digestions. The different tested prototypes had similar coating thickness but different in the percentage diol on the hetero-functional coating, which was controlled by the level of oxidation as described in Example 16. Table 6 below shows the difference in the performance of the said prototypes. Total ion recovery was higher for trypsin bioconjugated on a heterobifunctional coating with lower % diol to aldehyde conversion. The ratio between diol and aldehyde can be tuned to fit desired interaction between the surface and the analyte.

TABLE 6
Performance comparison prototypes with
different diol to aldehyde conversion

| | | | % Diol | | |
|---|---|---|---|---|---|
| | Aldehyde, | Trypsin, | con- | % Miss- | Total Ion |
| Products | μmol/m² | mg/g | version | cleavage, | Intensity |
| 18A | 2.50 | 30.0 | 21 | 7.2 | 2.29E+09 |
| 18B | 3.02 | 32.3 | 26 | 12.5 | 2.41E+09 |
| 18C | 3.30 | 32.6 | 29 | 10.3 | 2.30E+09 |
| 18D | 3.93 | 36.6 | 34 | 9.3 | 1.36E+09 |
| 18E | 4.58 | 38.1 | 40 | 8.8 | 1.69E+09 |
| 18F | 4.98 | 38.0 | 43 | 9.7 | 2.06E+09 |

While this disclosure has been particularly shown and described with reference to example embodiments thereof, it will be understood by those skilled in the art that various changes in form and details may be made therein without departing from the scope of the technology encompassed by the appended claims. For example, other chromatography systems or detection systems can be used.

1. A hetero-functional coating applied on a solid support, the coating comprising:
a first functionality for conjugating biomolecules for the analysis of a protein or nucleic acid, and
a second functionality for preventing undesired interactions between analytes of interest and the surface of solid support.

2. The hetero-functional coating of claim 1, wherein the ratio of the second functionality to the first functionality is at least 15% of a total surface coverage.

3. The hetero-functional coating of claim 1, wherein the total surface coverage is greater than 5 μmoles/m².

4. The hetero-functional coating of claim 1, wherein the second functionality is hydrophilic.

5. The hetero-functional coating of claim 4, wherein the second functionality comprises a diol to impart the hydrophilicity.

6. The hetero-functional coating of claim 1, wherein the first functionality is an aldehyde.

7. The hetero-functional coating of claim 1, wherein the conjugation of biomolecules is via reductive amination.

8. The hetero-functional coating of claim 1, wherein the first functionality is an epoxide.

9. The hetero-functional coating of claim 8, wherein the conjugation of biomolecules is via the epoxide.

10. The hetero-functional coating of claim 1, wherein the hetero-functional coating is hydrophilic that is activated by alkoxy silane.

11. The hetero-functional coating of claim 10, wherein the alkoxy silane is an aldehyde.

12. The hetero-functional coating of claim 11, wherein conjugating biomolecules comprises conjugating the biomolecules through the aldehyde via reductive amination.

13. The hetero-functional coating of claim 10, wherein the alkoxy silane is an acryloxy silane.

14. The hetero-functional coating of claim 13, wherein conjugating the biomolecules comprises conjugating the biomolecules through the acryloxy silane via Michael addition.

15. The hetero-functional coating of claim 10, wherein the alkoxy silane is an amine silane, further comprising reacting the amine silane with a diacrylate group or a dialdehyde group.

16. The hetero-functional coating of claim 15, wherein the immobilized biomolecules are reacted with the dialdehyde group via reductive amination.

17. The hetero-functional coating of claim 15, wherein the immobilized biomolecules are reacted with the diacrylate group via Michael addition.

18. The hetero-functional coating of claim 15, wherein the biomolecules are immobilized on the coating, and wherein the immobilized biomolecules are pre-modified with an aldehyde or an acrylate chemistry.

19. The hetero-functional coating of claim 1, wherein the biomolecules are immobilized on the hetero-functional coating.

20. The hetero-functional coating of claim 19, wherein the immobilized biomolecules comprise a single enzyme or mixed enzymes.

21. The hetero-functional coating of claim 20, wherein the enzyme is chosen from a group comprising protease, lipases, phospholipases, ligases, transferases, oxidoreductases, isomerases, hydrolases, or a mixture thereof.

22. The hetero-functional coating of claim 19, wherein the immobilized biomolecules are a single enzyme or a mixture of enzymes chosen from a group of a protease enzymes.

23. The hetero-functional coating of claim 22, wherein the protease enzymes are trypsin, Lyc-C, Asp-N, pepsin, Glu-C, or a mixture thereof.

24. The hetero-functional coating of claim 22,
wherein the protease enzyme is IdeZ or IdeS, and
wherein the enzyme is used in characterizing antibodies and antibody drug conjugates.

25. The hetero-functional coating of claim 20, wherein the enzyme is a glycosidase for O-glycan and N-glycan profiling and mixtures thereof.

26. The hetero-functional coating of claim 20, wherein the enzyme is from the family of glycosidase and is used for hydrolysis of glucuronides drug conjugates.

27. The hetero-functional coating of claim 19, wherein the immobilized biomolecules are an affinity ligand.

28. The hetero-functional coating of claim 27, wherein the affinity ligand is an immoglobin-binding protein.

29. The hetero-functional coating of claim 28, wherein the immoglobin-binding protein is protein A, G, L or a mixture thereof.

30. The hetero-functional coating of claim 27, wherein the affinity ligand is an antigen binding.

31. The hetero-functional coating of claim 30, wherein the antigen binding is an antibody, nanobody, or a mixture thereof.

32. The hetero-functional coating of claim 27, wherein the affinity ligand is an aptamers.

33. The hetero-functional coating of claim 27, wherein the affinity ligand contains avidin and is used with biotinylated protein samples.

34. The hetero-functional coating of claim 33, wherein the avidin containing affinity ligand is streptavidin, avidin, or neutravidin.

35. The hetero-functional coating of claim 19, wherein the immobilized biomolecules comprise at least one enzyme and/or at least one affinity ligand.

36-153. (canceled)
